(19) United States
(12) Patent Application Publication
(10) Pub. No.: US 2025/0176923 A1
(43) Pub. Date: Jun. 5, 2025

(54) Cognitive Artificial Intelligence Platform for Physicians

(71) Applicant: Elevance Health, Inc., Indianapolis, IN (US)

(72) Inventors: Moova Nageshwara Rao, Hyderabad (IN); Aditya Gautam Surampudi, Hyderabad (IN); Krishna Chaitanya Gollapudi, Hyderabad (IN)

(73) Assignee: Elevance Health, Inc., Indianapolis, IN (US)

(21) Appl. No.: 18/962,869

(22) Filed: Nov. 27, 2024

Related U.S. Application Data
(60) Provisional application No. 63/604,348, filed on Nov. 30, 2023.

(51) Int. Cl. A61B 5/00; G06F 40/40; G16H 10/60

(52) U.S. Cl. CPC A61B 5/743; G06F 40/40; G16H 10/60

(57) ABSTRACT

The computer-implemented system includes a computer having a processor and memory with executable instructions that run a platform for creating and displaying medical information. The system may receive medical data relating to a patient in FHIR/LPR format, including patient records and vital statistics. The system may perform temporal analysis and feature extraction on the medical data. Using an artificial intelligence model trained on historical patient data, the system may analyze the data to generate a knowledge graph by identifying nodes, determining relationships using semantic analysis and natural language processing, generating edges, and applying graph database algorithms. The system may create a visual output including a health snapshot with generated prose ranked using page rank algorithms, an interactive body map identifying health issues, and an enhanced timeline format health history with drill-down capabilities.

### CROSS-REFERENCE TO RELATED APPLICATIONS

This application claims priority to and the benefit of U.S. Provisional Application No. 63/604,348, filed Nov. 30, 2023, the entirety of which is incorporated herein by reference.

### TECHNICAL FIELD

The disclosed implementations relate generally to the use of artificial intelligence to analyze heterogeneous sets of data and relate more specifically to the use of artificial intelligence to analyze medical records to present the results of the analysis in an enhanced format and with enhanced interpretations of the data.

### BACKGROUND

Electronic health records, as they exist and are created by providers across the health care marketplace, are not reader-friendly for physicians, others who assist physicians, and other professionals and staff members, whose jobs include review of medical records for their patients. Records being in non-standard, fragmented formats makes review of historical encounters difficult, which is an important part of preliminary diagnoses.

It is therefore desirable to build machine learning models on fragmentary data, such as the data that populate electronic medical records, to enhance readability of the data and to expand the set of data to create additional information including predictions, statistical analysis, etc. A platform to present the results of the machine learning and artificial intelligence analysis and data enhancement is also desirable.

### SUMMARY

A system of one or more computers can be configured to perform particular operations or actions by virtue of having software, firmware, hardware, or a combination of them installed on the system that in operation causes the system to perform the actions. One or more computer programs can be configured to perform particular operations or actions by virtue of including instructions that, when executed by data processing apparatus, cause the apparatus to perform the actions. One general aspect includes a computer-implemented system.

The computer-implemented system also includes a computer having a processor and a memory, the memory having stored thereon computer executable instructions that, when executed by the processor, cause the processor to run a platform for creating and displaying medical information. The system may receive medical data relating to a patient, where the medical data may include a patient record and at least one vital statistic. The system may perform temporal analysis on the medical data to produce temporal analysis data. The system may extract features of the medical data to produce features data. The system may use an artificial intelligence model, analyze the medical data, the temporal analysis data and the features data to create a visual output that may include information from the medical data, in an enhanced graphical format. In some embodiments, the artificial intelligence model is trained using a corpus of archival patient data relating to a plurality of prior patients.

In some embodiments, the system may receive medical data relating to a patient in FHIR/LPR format, including patient records and vital statistics. The system may perform temporal analysis and feature extraction on the medical data. Using an artificial intelligence model trained on historical patient data, the system may analyze the data to generate a knowledge graph by identifying nodes, determining relationships using semantic analysis and natural language processing, generating edges, and/or applying graph database algorithms. The system may create a visual output comprising: a health snapshot with automatically generated prose ranked using page rank algorithms, an interactive body map identifying health issues, and an enhanced timeline format health history with drill-down capabilities.

Other embodiments of this aspect include corresponding computer systems, apparatus, and computer programs that are recorded on one or more computer storage devices, each configured to perform the actions of the methods.

Implementations may include one or more of the following features. The platform may be further configured to create, using the artificial intelligence model, a knowledge graph from the medical data, the temporal analysis data and the features data.

The visual output may include information relating to a prediction generated by the artificial intelligence model based on the medical data, the temporal analysis data, and the features data, where the prediction relates to a factor identified by the probabilistic artificial intelligence model as having a substantial correlation to the at least one subject in the plurality of prior patients.

The enhanced graphical format may include a health snapshot, where the health snapshot contains automatically generated prose relating to the patient's health.

The graphical format may include a health history. The health history is displayed in a timeline format.

The graphical format may include a body map, and where the body map identifies at least one area of the patient's body where the patient record indicates at least one of an existing health problem, a previous health problem, and a predicted health problem.

The platform may be further configured to generate at least one recommendation relating to the patient.

The platform may be further configured to calculate and display a health score using the probabilistic artificial intelligence model and the medical data.

The prediction may relate to a risk of a negative drug outcome, and where the archival patient data includes drug prescription data and negative drug outcome data, and where the artificial intelligence module is configured to identify at least one feature of the archival patient data that correlates with a negative drug outcome.

The prediction may relate to a risk of the patient contracting a specified disease.

The medical data further may include a medical image, the platform being further configured to use the probabilistic artificial intelligence model to analyze the image to identify at least one portion of the image that indicates a likelihood of a diagnosis, where the analysis is based on a correlation between patients in the plurality of prior patients having the diagnosis, and similarly appearing portions of images of the same organ or body part as the medical image.

In some embodiments, the visual output may include predictions generated by the artificial intelligence model based on knowledge graph analysis. The system may calculate and display health scores using the knowledge graph and artificial intelligence model. The knowledge graph can represent prescribed medications and negative drug outcomes as nodes, enabling drug-related risk factor identification. For medical images, the knowledge graph may include nodes representing image features and diagnostic correlations. The system may extract patient information about associated organizations, payors, and practitioners as nodes. The page rank algorithm may rank health information based on reference frequency in medical records. The body map may display interactive indicators as dots on specific body parts, revealing detailed health information upon selection. The artificial intelligence model may analyze medical images to identify diagnosis-correlated features based on historical data. The system may generate confidence scores for predicted diagnoses based on similarity between current and historical features.

Implementations of the described techniques may include hardware, a method or process, or computer software on a computer-accessible medium.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is a block diagram of a system in accordance with one aspect of the present disclosure.

FIG. 2A is an architectural overview of a system for a cognitive artificial intelligence platform, for use in patient record searches, in accordance with one aspect of the present disclosure.

FIG. 2B is an exemplary user interface in accordance with one aspect of the present disclosure.

FIG. 2C is an exemplary knowledge graph in accordance with one aspect of the present disclosure.

FIG. 2D is a block diagram of data structures for use in one aspect of the present disclosure.

FIG. 3 is an architectural overview of a system for a cognitive artificial intelligence platform, for use in predicting negative outcomes from prescribing drugs, in accordance with one aspect of the present disclosure.

FIG. 4A an architectural overview of a system for a cognitive artificial intelligence platform, for use in predictions, in accordance with one aspect of the present disclosure.

FIG. 4B is an exemplary user interface in accordance with one aspect of the present disclosure.

FIG. 5 is a flow diagram of a method in accordance with one aspect of the present disclosure.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if”' is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

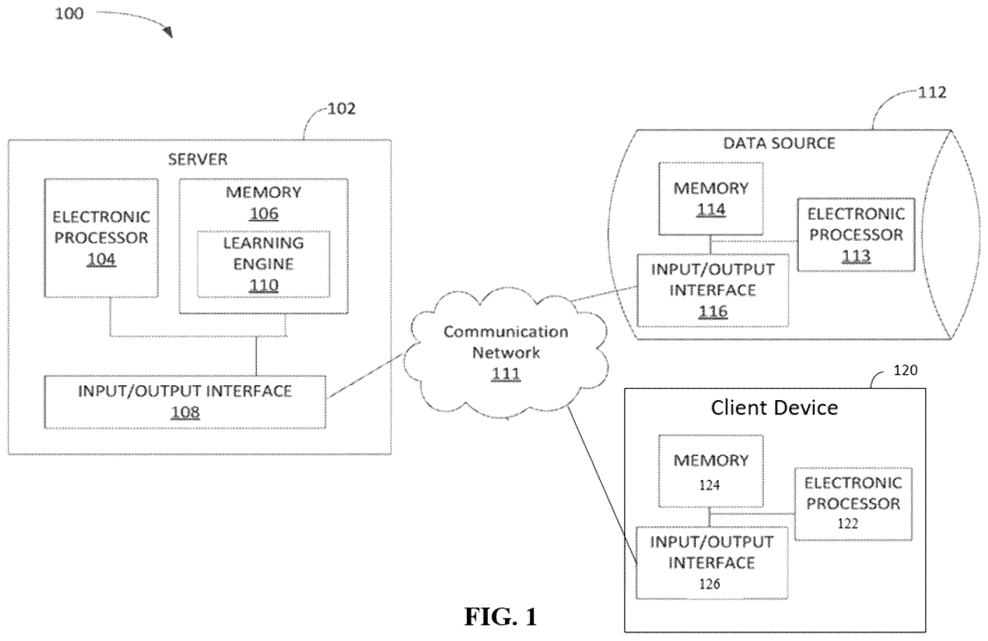

FIG. 1 illustrates a system 100 for creating, running, and serving a platform for analysis and display of medical records enhanced by artificial intelligence, according to some embodiments of the invention. The system 100 includes a server 102 that includes a plurality of electrical and electronic components that provide power, operational control, and protection of the components within the server 102. For example, as illustrated in FIG. 1, the server 102 may include an electronic processor 104 (e.g., a microprocessor, application-specific integrated circuit (ASIC), or another suitable electronic device), a memory 106 (e.g., a non-transitory, computer-readable storage medium), and an input/output interface 108. The electronic processor 104, the memory 106, and the input/output interface 108 communicate over one or more connections or buses. The server 102 illustrated in FIG. 1 represents one example of a server, and embodiments described herein may include a server with additional, fewer, or different components than the server 102 illustrated in FIG. 1. Also, in some embodiments, the server 102 performs functionality in addition to the functionality described herein. Similarly, the functionality performed by the server 102 (i.e., through execution of instructions by the electronic processor 104) may be distributed among multiple servers. Accordingly, functionality described herein as being performed by the electronic processor 104 may be performed by one or more electronic processors included in the server 102, external to the server 102, or a combination thereof.

The memory 106 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof, which may include transitory memory, non-transitory memory, or both. The electronic processor 104 executes computer-readable instructions (“software”) stored in the memory 106. The software may include firmware, one or more applications, program data, filters, rules, one or more program modules, and other executable instructions. For example, the software may include instructions and associated data for performing the methods described herein. For example, as illustrated in FIG. 1, the memory 106 may store a learning engine (e.g., “software”) 110 for performing one or more of the functions described herein, which may include probabilistic matching, deterministic matching, machine learning, artificial intelligence, or the like. However, in other embodiments, the functionality described herein as being performed by the learning engine 110 may be performed through one or more software modules stored in the memory 106 or external memory.

The input/output interface 108 allows the server 102 to communicate with devices external to the server 102. For example, as illustrated in FIG. 1, the server 102 may communicate with one or more data sources 112 through the input/output interface 108. In particular, the input/output interface 108 may include a port for receiving a wired connection to an external device (e.g., a universal serial bus (“USB”) cable and the like), a transceiver for establishing a wireless connection to an external device (e.g., over one or more communication networks 111, such as the Internet, a local area network (“LAN”), a wide area network (“WAN”), and the like), or a combination thereof.

In some embodiments, the server 102 also receives input from one or more peripheral devices, such as a keyboard, a pointing device (e.g., a mouse), buttons on a touch screen, a scroll ball, mechanical buttons, and the like through the input/output interface 108. Similarly, in some embodiments, the server 102 provides output to one or more peripheral devices, such as a display device (e.g., a liquid crystal display (“LCD”), a touch screen, and the like), a printer, a speaker, and the like through the input/output interface 108. In some embodiments, output may be provided within a graphical user interface (“GUI”) (e.g., generated by the electronic processor 104 executing instructions and data stored in the memory 106 and presented on a touch screen or other display) that enables a user to interact with the server 102. In other embodiments, a user may interact with the server 102 through one or more intermediary devices, such as a personal computing device, e.g., laptop, desktop, tablet, smartphone, smartwatch or other wearable device, smart television, and the like. For example, a user may configure functionality performed by the server 102 as described herein by providing data to an intermediary device that communicates with the server 102. In particular, a user may use a browser application executed by an intermediary device to access a web page that receives input from and provides output to the user for configuring the functionality performed by the server 102.

As illustrated in FIG. 1, the system 100 includes one or more data sources 112. Each data source 112 may include a plurality of electrical and electronic components that provide power, operational control, and protection of the components within the data source 112. In some embodiments, each data source 112 represents a server, a database, a personal computing device, or a combination thereof. For example, as illustrated in FIG. 1, each data source 112 may include an electronic processor 113 (e.g., a microprocessor, ASIC, or other suitable electronic device), a memory 114 (e.g., a non-transitory, computer-readable storage medium), and an input/output interface 116. The data sources 112 illustrated in FIG. 1 represent one example of data sources and embodiments described herein and may include a data source with additional, fewer, or different components than the data sources 112 illustrated in FIG. 1. Also, in some embodiments, the server 102 communicates with more or fewer data sources 112 than illustrated in FIG. 1.

The input/output interface 116 allows the data source 112 to communicate with external devices, such as the server 102. For example, as illustrated in FIG. 1, the input/output interface 116 may include a transceiver for establishing a wireless connection to the server 102 or other devices through the communication network 111 described above. Alternatively or in addition, the input/output interface 116 may include a port for receiving a wired connection to the server 102 or other devices. Furthermore, in some embodiments, the data sources 112 also communicate with one or more peripheral devices through the input/output interface 116 for receiving input from a user, providing output to a user, or a combination thereof. In other embodiments, one or more of the data sources 112 may communicate with the server 102 through one or more intermediary devices. Also, in some embodiments, one or more of the data sources 112 may be included in the server 102.

The memory 114 of each data source 112 may store patient data, such as medical records, and the like. For example, the data sources 112 may include an electronic medical record (“EMR”) database, a claims database, a patient database, and the like. In some embodiments, as noted above, data stored in the data sources 112 or a portion thereof may be stored locally on the server 102 (e.g., in the memory 106).

User device 120 may also be connected to communication network 111, for communication with server 102 and/or with data source 112. Inputs and outputs 118 may flow between server 102, e.g., via input/output interface 108, and user device 120, e.g., via input/output interface 126. Inputs may include medical records data as described herein, including diagnosis data, medical images, patient biographical data, historical timeline data, and the like. Outputs may include match determinations via probabilistic matching, deterministic matching, and/or machine learning, as described in more detail below.

A cognitive artificial intelligence platform for medical records is herein disclosed, to process and build machine learning model(s) from electronic health records. Electronic health records may include vital statistics, laboratory reports, medical images, and video or audio records reflecting patient episodes. Other medical records may also be included. Medical records may in some embodiments be embedded in Fast Healthcare Interoperability Resources (FHIR)/Longitudinal Patient Records (LPR) files, which may be stored as a Javascript Object Notation (“JSON”) data structure.

A cognitive artificial intelligence platform, as herein disclosed, may process an LPR into meaningful descriptive language and infographics relating to historical episodes of a patient, using a graph database (e.g., GraphDB). A comprehensive visualization platform reduces cognitive load and review burden for health care providers, e.g., during prescreening of patients. A platform as disclosed may also provide scope for sample selection for building machine learning models. The data available in GraphDB may also establish similarities in population on various features that may appear in a health record such as an LPR. Transformation of data from electronic health records, such as FHIR records, into structured data may allow a reviewer to focus on what is most important, by identifying the important information in the records and then highlighting it graphically during review. A platform as disclosed herein may also perform selective points storage and establish relationships across the population. These features may facilitate ease of sample selection for models and ease of understanding to health care providers.

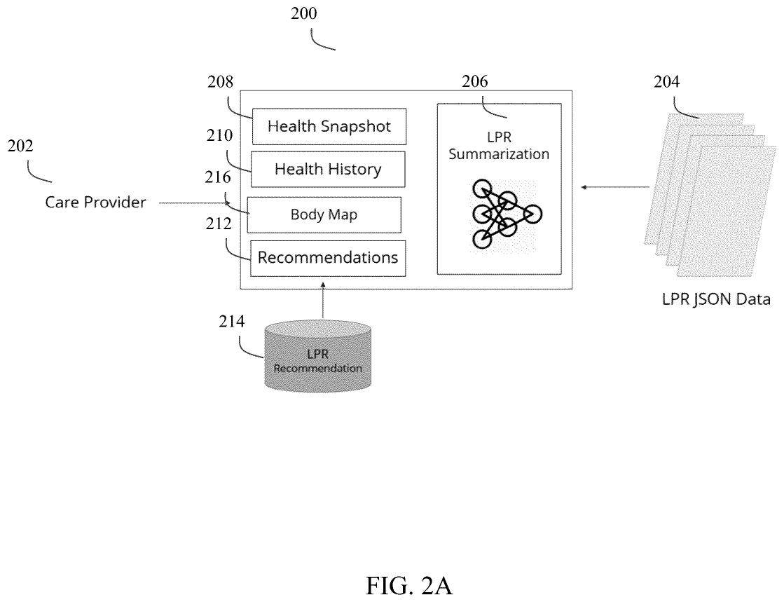

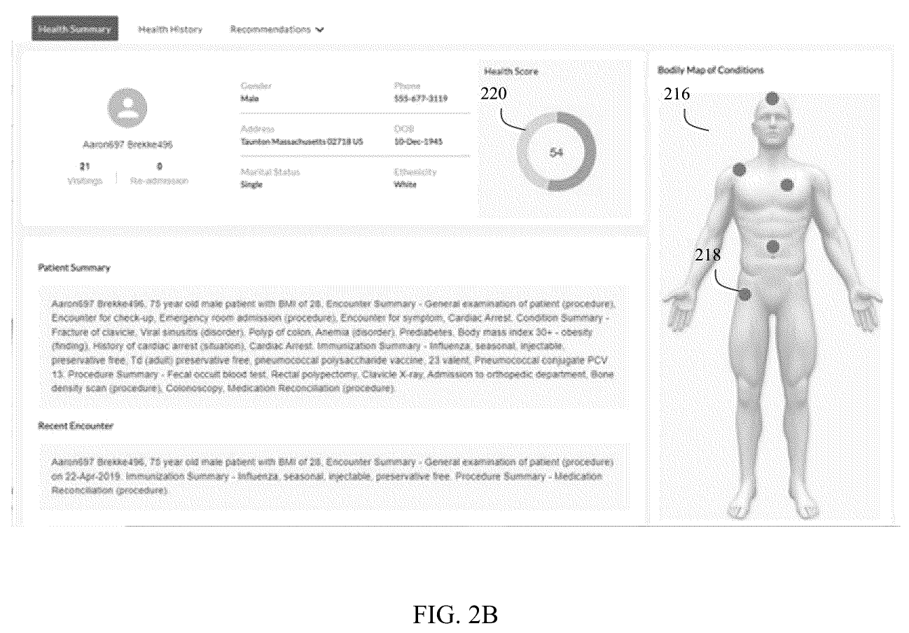

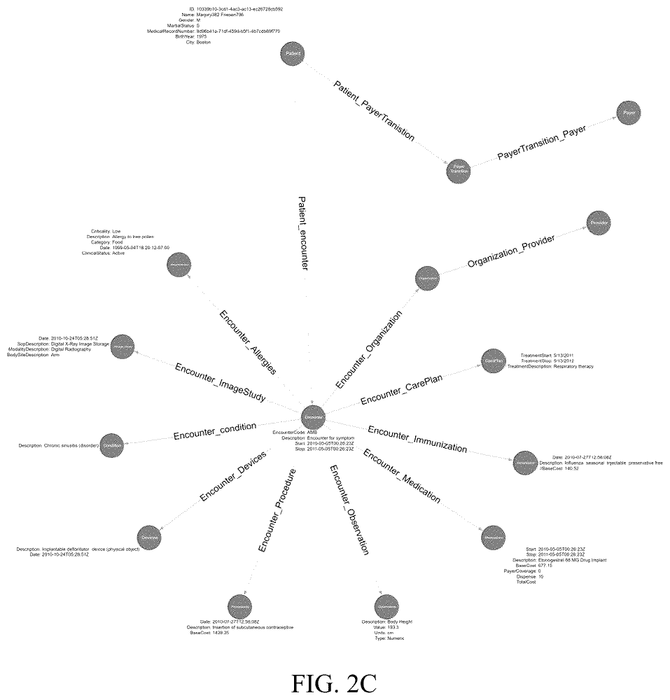

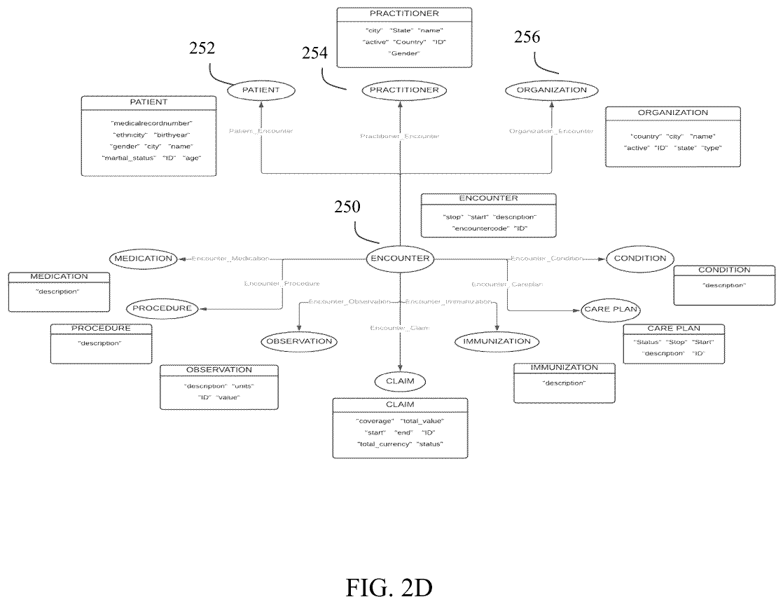

Turning now to FIG. 2A, an architectural overview of a system 200 for a cognitive artificial intelligence platform, for use in a patient record search, is shown. As noted above, health care records exist in heterogeneous and sometimes haphazard data formats. They may exist as flat text, images, etc., with each record for a given patient disconnected from the others. Artificial intelligence may be used, in accordance with one aspect of the present disclosure, to effectively display and demonstrate an LPR. Care provider 202 may use system 200 to access LPR 204, which may in some embodiments be stored as JSON data. System 200 may perform certain operations upon LPR 204, which may include LPR summarization 206. LPR summarization 206 may include the use of artificial intelligence, including language models, to create a health snapshot 208 of the patient's health record. Health snapshot 208 may in some embodiments be in a textual format such as prose. Health snapshot 208, e.g., prose, may in some embodiments be shorter to read and more generally at a higher level than review of the individual entries in LPR 204. An exemplar of health snapshot 208 is shown in FIG. 2B.

In some embodiments, health snapshot 208 may be developed using an artificial intelligence algorithm. In some embodiments, the artificial intelligence algorithm for developing a prose snapshot such as health snapshot 208 may include the use of a page rank algorithm. A page rank algorithm, such as that used in modern search engine algorithms, may in some embodiments be based on a ranking of a page based on how many other pages link to the initial page. In some embodiments, a page rank algorithm may be utilized to select the most linked medical records, from the patient's other medical records, and include more or less information from those pages, based on a calculated rank, in a prose summary such as health snapshot 208.

In some embodiments, health snapshot 208 may be developed with heuristics rather than or in addition to artificial intelligence. Heuristics may include generating a summary based on previous summaries that may have been manually written, or that may have been automatically generated previously and may in some instances have been identified as valuable by a reviewer such as a physician.

Returning to FIG. 2A, system 200 may also create a health history 210 from the LPR 204. Health history 210 may be displayed using enhanced graphics, e.g., a timeline display. Health history 210 may be created using artificial intelligence analysis on each element of the LPR 204, to determine the relevant data to be placed in the history, e.g., the date of an encounter or a diagnosis or the relevant words to describe the encounter or diagnosis.

Operations performed by system 200 may also include creation of one or more health recommendations 212, which may call upon historical data 214 that is collected for use in health recommendations. Health recommendations 212 may be generated using artificial intelligence analysis using an artificial intelligence model that may be trained on the historical data 214. In some embodiments, the artificial intelligence may analyze the historical data 214 to determine one or more correlations between specific treatments for specific health issues, and success at managing or curing the health issue. In some embodiments, historical data may be tagged with health attributes that may be identified as useful or informative. The presence of the same attributes in an LPR 204 being evaluated may be used to inform the display, including, e.g., a recommendation, a prediction, or a health score as discussed below.

System 200 may also use artificial intelligence to create a body map 216. An exemplary body map is shown in FIG. 2B, which may be displayed by system 200. In some embodiments, an artificial intelligence model may analyze LPR 204 to extract organ systems, body parts, etc. where the patient may have, or have had, a health issue. In some embodiments, an artificial intelligence model may be trained to associate certain health issues with certain body parts or organ systems, based on the name or other information relating to the health issue, even if the names of related body parts or organ systems are not identified in LPR 204 in relation to the issue. The interactive indicators may be displayed as dots on specific body parts, and selecting an indicator may reveal detailed health information for the corresponding body part. In response to identifying certain health issues for certain body parts or organs, the artificial intelligence model may assign an indicator 218, such as a dot, to the particular portion of the human representative in the body map image that corresponds to the certain body part or organ. In some embodiments, a user action, such as hovering over indicator 218, may reveal further information, such as alternate text, that identifies the body part, organ system, health issue, or any of the above. In some embodiments, a second user action, such as clicking, on indicator 218 may reveal further details, and may navigate away from the body map to a more detailed view of information relating to the body part, organ system, or health issue represented on body map 216 by indicator 218.

FIG. 2B shows an exemplary user interface as generated by system 200 of FIG. 2A. As discussed above, the user interface may include a health snapshot 208 and a body map 216, both of which may be generated by system 200 via an artificial intelligence model analyzing a patient LPR 204, in some instances also analyzing, and/or being trained by, historical data 214.

FIG. 2B also shows a health score 220, which may be computed by the artificial intelligence model based on training data. In some embodiments, health score 220 may be the result of predictive artificial intelligence, which may analyze and be trained upon historical data 214 to predict a correlation between certain health conditions, or other factors such as vital statistics, and good or ill health, based on an existence of such a correlation in historical data 214. In some embodiments, the health score may evaluate two descriptions of conditions for identified factors relating to similarity, and then use the similarity data generated by the evaluation to calculate health score 220.

Analysis of LPR 204, e.g., by an artificial intelligence model, may make use of a graph database, such as knowledge graphs. In some embodiments, a graph database may be a database that utilizes a graph structure for semantic queries with nodes, edges, and properties to represent stored data. The graph structure relates data items to a collection of nodes and edges, the edges representing relationships between the nodes. The relationships allow data to be linked together directly and, in many cases, retrieved with one operation. Leveraging relationships within a graph database can be fast for artificial intelligence models because the relationships are stored within the database itself. Relationships can be intuitively visualized using the graph database, making the relationships useful for heavily interconnected data.

A knowledge graph is a knowledgebase integrated with a graph database. By integrating a knowledgebase with a graph database, a knowledge graph supports a much wider and deeper range of services than a standard graph database. In other words, the knowledge graph links information together, such as, for example, facts, entities, and locations, to create interconnected search results that are more accurate and relevant. More specifically, the knowledge graph is a knowledgebase consisting of millions of pieces of data corresponding to specific patient information and the context or intent behind asking for the information based on available content. Complex associated information can be better inquired by using a knowledge graph. An exemplary knowledge graph is shown in FIG. 2C.

One or more artificial intelligence models may generate a knowledge graph using semantic analysis of the LPR 204 or other health care records materials. Artificial intelligence models for generating a knowledge graph using semantic analysis may include one or more graph database algorithms, which may in some embodiments be used to ascertain a relationship, represented by an edge, between two entries in LPR 204, e.g., two encounters or diagnoses, represented by nodes. Natural Language Processing Text Rank algorithms may also be used, in some embodiments, to extract keywords and rank phrases. Text similarity and summarization algorithms may also be used, in some embodiments, to compare different passages of text found in LPR 204, and/or to create summaries of lengthier passages of text found in medical records, such as LPR 204.

An example of a knowledge graph that may be created from a longitudinal patient record such as LPR 204, is a drug abuse model. In a graph of a drug abuse model, drugs themselves can be represented as nodes of the graph. Exposure to particular drugs by particular patients may be represented in a knowledge graph, as an edge. Using a positive drug abuse LPR member, the relationship with other similar members can be established and their features can be used to create and/or train an artificial intelligence model, which would allow for predictions to be generated regarding drug dependency based on particular drugs and other health factors in the patient's LPR.

Initially, the artificial intelligence model or models may receive a knowledge graph data, such as a knowledge base data structure. The basic knowledge base may be generated from semantic analysis of the LPR 204 or other health care records materials. The artificial intelligence model may extract data elements from the knowledgebase data structure that represent the nodes of the knowledge graph. The data elements may be different medical-related topics regarding the patient. The artificial intelligence model may then identify a relationship between two different data elements by analyzing the LPR 204 or other health care records materials using the artificial intelligence model and one or more model characteristics. The artificial intelligence model may then determine whether the relationship meets certain relationship criteria, such as whether the relationship between the two different data elements has a high correlation based on comparisons to historical health care data. In response to determining that the relationship meets certain relationship criteria, the artificial intelligence model may generate an edge between the two different data elements. After the artificial intelligence model has analyzed the knowledge base data structure and generated the nodes and edges, a visualized representation of the knowledge graph may be displayed on a user interface, such as the exemplary knowledge graph shown in FIG. 2C.

The knowledge graph may be generated by (i) identifying data elements as nodes in the knowledge graph by extracting them from the knowledgebase data structure, where the data elements represent different medical-related topics regarding the patient; (ii) determining relationships between the data elements using semantic analysis and natural language processing of the medical data; (iii) generating edges between the nodes based on the determined relationships when they meet certain relationship criteria, such as having a high correlation based on comparisons to historical health care data; and/or (iv) applying graph database algorithms to ascertain relationships represented by the edges between entries in the medical data.

A node of a knowledge graph may be a data structure relating to a health, identity, or other factor relating to a patient. Nodes may be visually represented by circles as shown in FIG. 2C. Examples of nodes may include a device, such as an implantable defibrillator, a condition, such as a chronic disorder or an allergy, with which the patient has been diagnosed, or a procedure which the patient underwent, a test the patient had, a treatment the patient underwent, a medication the patient has been prescribed, or one or more vital statistics measured at a given time. Nodes may also be used to represent the patient him or herself, one or more organizations with which the patient is associated, e.g., an employer, or one or more responsible payors who may be responsible for the patient's medical expenses, e.g., a parent or guardian.

An edge of the graph, represented in FIG. 2C by a line connecting two or more nodes, may represent a relationship between the two nodes, which may in some embodiments be represented by a health encounter. For example, a patient may have had a health care encounter, e.g., a doctor's appointment, where the patient presented with a symptom. The patient may have had vital statistics measured, may have had one or more tests done, e.g., imaging, and may have ended up prescribed one or more medications or devices. This encounter node may be linked, by edges, to the diagnoses, the tests, the vital statistics, etc. The encounter may then also be linked to the persons or entities through whom the patient obtained insurance, the payors of the medical expenses, etc.

In some embodiments, LPR data, e.g., for use in creating a knowledge graph, may reside as a JavaScript Object Notation (“JSON”) object in a Fast Health Interoperability Resources (“FHIR”) format, which is known in the art as a way of storing the data.

Turning now to FIG. 2D, exemplary data structures are shown to identify different commonly used entities and entity types that may appear frequently in medical records such as LPR 204. In some embodiments, a tree of data structures centered around encounters as shown in FIG. 2D, may be used for graph algorithms to obtain or compute semantic relatability of nodes. At the center of the tree is the encounter data structure 250, which may include information about each health care encounter in LPR 204. Encounter data structure 250 may include information about the encounter, such as its start and stop dates. Encounter data structure 250 may then contain or otherwise be associated with other data structures relating to the kinds of encounter that occurred. A patient data structure 252 may include data about the patient as shown, which may include a record number and biographical information. Practitioner data structure 254 and organization data structure 256 may include similar kinds of information as the patient data structure 252, to identify a health care practitioner or a health care organization. Data structures may also exist for other identifying features of an encounter, such as a medication, a procedure, an observation, an insurance claim, an immunization, a care plan, or a condition. Data structures such as these may be built or populated by system 200 to structure medical record data and may be used in the display of the data, such as health snapshot 208 or body map 216, or subsequent user interface interactions relating to either.

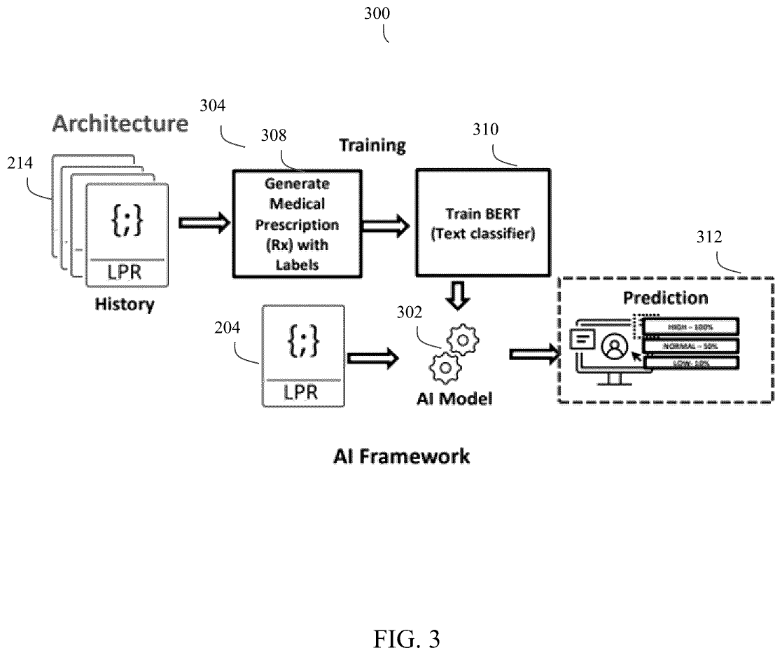

Turning now to FIG. 3, an architectural overview of a system 300 for a cognitive artificial intelligence platform for the enhancement of medical records data is shown. The system 300 shown in FIG. 3 may be used to assess risk of drug abuse, additions, overdoses, or other issues resulting from prescription of certain drugs (e.g., opioids) to the patient whose medical records are being reviewed. System 300 may include an artificial intelligence model 302, which may be trained using training data 304. Training data 304 may include a population of historical patient records, e.g., historical data 214. Historical data 214 may then be evaluated to extract and generate medical prescription events 308, which may help identify which patients in the set of historical data 214 may have been prescribed drugs that carry a risk of addiction or overdose. A text classifier 310 may also be used to evaluate the historical data 214. Bidirectional Encoder Representations from Transformers (“BERT”) is an example of a text classifier 310 that may be used to locate drug prescriptions in patient records by evaluating text. The output of text classifier 310 may then be fed to artificial intelligence model 302 for training. Training may in some embodiments include identifying features in medical records where negative drug-related events occurred and determining the extent of any correlation between the existence of certain features and the negative event.

The patient's record, e.g., LPR 204 as shown in FIG. 2A, may also be input to artificial intelligence model 302, as trained by training data 304. Artificial intelligence model 302 may then evaluate LPR 204 in view of the training data, including events it may have identified as having a significant correlation to negative drug outcomes such as addiction or abuse. Artificial intelligence model 302 may output a prediction 312 as to the risk of a negative drug outcome. In some embodiments, the prediction may be displayed on the platform, and may be displayed as a classification, such as low, medium, or high risk.

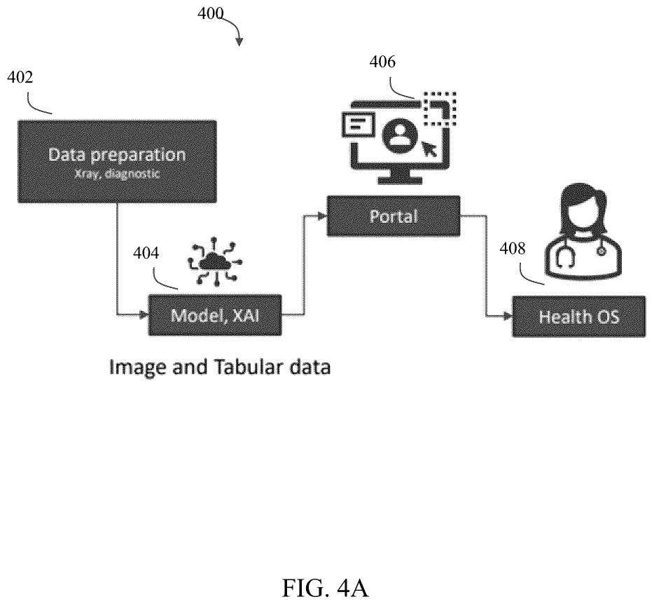

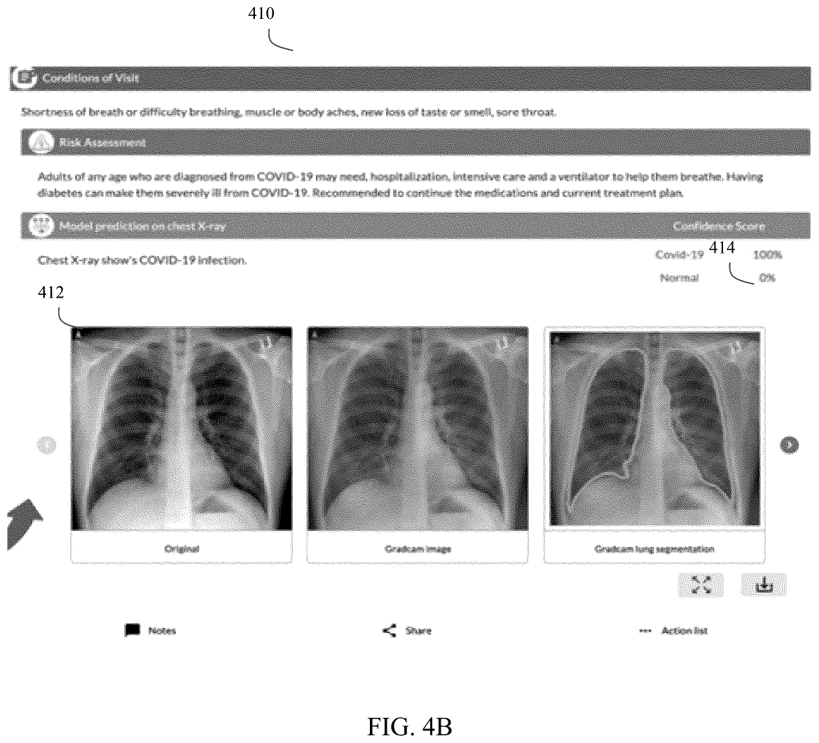

Turning now to FIG. 4A, an information flow diagram of an architecture for a system 400 for disease predictions is shown. System 400 may include data preparation 402 for patient records (e.g., LPR 204 of FIG. 2A). Patient records may include diagnostic test results, imaging, x-rays, or the like. Data preparation may include data pre-processing, which may include LPR flattening reusable scripts, to transform data as a table structure & unstructured data as files, e.g., JPEG files. In some embodiments, data may be extracted from LPR 204, which may be stored in a JSON format. Images found in LPR 204 may be base64encoded, and may contain, for example, radiology imaging. Image files may then be written and standardized, e.g., to a standard shape and/or size, such as 299×299 pixels.

Once the data is prepared, an explainable artificial intelligence (“XAI”) model 404 may then evaluate the data. In some embodiments, XAI model 404 may be, or include, explainable artificial intelligence with Shapley Additive Explanation (“SHAP”), which may be used to identify positively contributing features to predict. The architecture of XAI model 404 may, in some embodiments, include the use of a Convolutional Neural Network (“CNN”). In some embodiments, the CNN may be implemented using a two dimensional convolution layer, e.g., a Conv2D layer.

In some embodiments, an XAI model may be trained using medical guideline information and admission and medical records and identify factors that most highly correlate with disease indicators such as readmission to a hospital. In some embodiments, training schema may include the use of supervised algorithms. XAI model 404 may then generate a risk prediction algorithm for a patient, which may include identified factors, which may include expenses, age, prescription information, and prior hospitalization information. Risk predictions may then be published to portal 406, which may then also be accessible via a health operating system 408.

Publication of disease prediction results on portal 406 may include presentation of the prediction graphically. Disease prediction via XAI model 404 may also include analysis of medical images that may be included in medical records, e.g., x-rays, scans, or other kinds of imaging. XAI model 404 may evaluate a medical image and identify features of the image that may not be present in typical images of the same organ system or body part, but which do appear on a substantial number of other patient records. XAI model 404 may in some embodiments be trained using historical records of other patients. Through training on such material, if sufficiently voluminous, XAI model 404 may be able to identify features of images whose presence or absence correlates to a specific diagnosis or the absence of a specific diagnosis, in a sufficient proportion of the historical patient population that the correlation should be considered when evaluating another patient whose images present the same feature. In response to identifying such features, the XAI model 404 may highlight portions of the medical image that correspond to the identified features.

FIG. 4B is an exemplary user interface 410 of a platform in accordance with one aspect of the present disclosure. FIG. 4B shows an image 412 of a set of lungs that was obtained via a chest x-ray. The image 412 is reproduced three times in user interface 410. The first reproduction is the original unaltered image of the lungs. The second and third reproductions are colored in such a way as to highlight parts of the lungs that may appear similar to some historical images of other patients' lungs, classified as having a particular disease or not, but different from others, such that the differences between them have been determined, e.g., by XAI model 404, to correlate with a diagnosis of a disease, e.g., COVID-19 for the purposes of the example in FIG. 4B. In some embodiments, a confidence score 414 may be included, as different features may have different levels of correlation with a given disease. A confidence score 414 may be calculated by XAI model 404 in view of historical data that may be analyzed to determine a level of correlation. Confidence score 414 may also be a function of a similarity determination, e.g., made by XAI model 404, between the identified feature of image 412, and the feature of the historical images that has been determined to be correlated with the diagnosis.

Similarly to medical images, in some aspects of the invention, an image of a set of lab results, e.g., numerical test results, may also be highlighted to bring the reviewer's attention to lab results that may be determined, from review of historical data by XAI model 404, to correlate with a diagnosis.

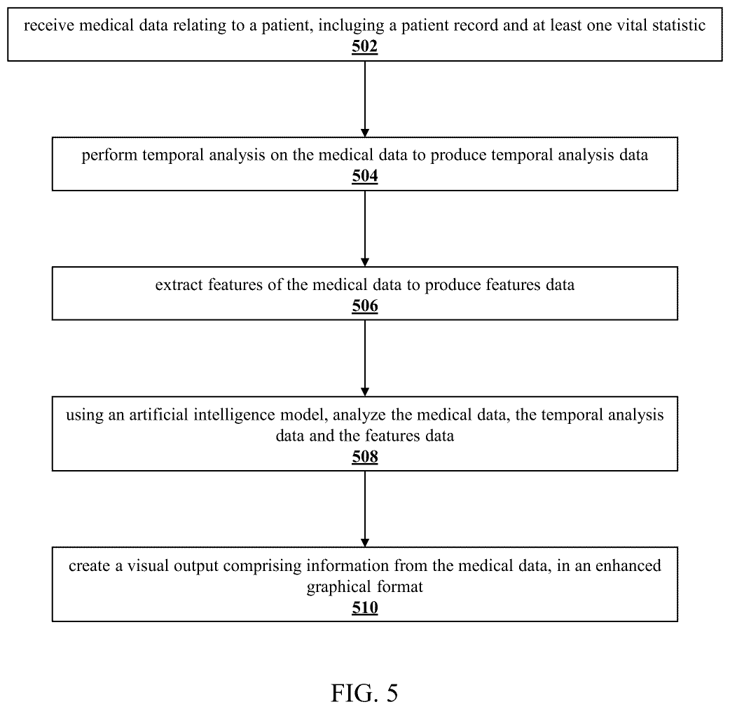

Turning now to FIG. 5, a flow chart of a method 500 of running a platform for display of medical information enhanced by cognitive artificial intelligence, in accordance with one aspect of the present disclosure, is shown. A system for running a platform, such as system 200 of FIG. 2A, or system 300 of FIG. 3, receives (502) medical data relating to a patient. The medical data may include a patient record and may also include a vital statistic. In some embodiments, the medical data may be included in a longitudinal patient record such as LPR 204. In some embodiments, as discussed above, the medical information may also include one or more medical images such as image 412 of FIG. 4B.

Temporal analysis is then performed (504) on the medical information. In some embodiments, the temporal analysis may include mining the medical data for date and time information, which may in some embodiments be used to create a timeline display such as health history 210. In some embodiments the timeline display created by the temporal analysis may allow for further detail to be displayed upon interaction with the timeline display by a user.

Features data may then be extracted (506) from the medical data, to produce features data. Features data may be any identified medical feature of the medical data relating to the patient, which may include, but are not limited to, encounters, medications, procedures, observations, claims, immunizations, care plans, or conditions. Features may also be associated with a patient, a practitioner, or an organization. Feature information that may be gleaned from feature extraction may be used by the platform to inform graphical outputs such as health snapshot 208, health history 210, or body map 216.

Medical data, temporal analysis data, and features data may then be analyzed (508) using an artificial intelligence model such as LPR summarization 206 or artificial intelligence model 302, or XAI model 404. As discussed above, analysis by the artificial intelligence model may be used to create a knowledge graph. Analysis by the artificial intelligence model may also be used to generate a recommendation, to generate data to be used on a body map such as body map 216, to generate a health history or a timeline such as health history 210, to calculate a health score such as health score 220, to calculate risk of a specific disease, to calculate a risk of drug addiction or other negative drug outcomes, or to analyze medical images, such as image 412, for useful diagnostic material.

A visual output may then be created (510) for transmission or display within the platform. As discussed here, a visual output may include a prediction, a health snapshot 208, a health history 210 that may include an interactive timeline, a body map 216, a health recommendation 212, or an annotated image 412.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

What is claimed is:

1. A computer-implemented system comprising
a computer having a processor and a memory, the memory having stored thereon computer-executable instructions that, when executed by the processor, cause the processor to run a platform for creating and displaying medical information, the platform being configured to:
receive medical data relating to a patient in a Fast Healthcare Interoperability Resources (FHIR) or Longitudinal Patient Records (LPR) format, wherein the medical data comprises a patient record and at least one vital statistic;
perform temporal analysis on the medical data to produce temporal analysis data;
extract features of the medical data to produce features data;
using an artificial intelligence model trained on historical patient data, analyze the medical data, the temporal analysis data and the features data to generate a knowledge graph by: (i) identifying data elements as nodes in the knowledge graph, (ii) determining relationships between the data elements using semantic analysis and natural language processing of the medical data, (iii) generating edges between the nodes based on the determined relationships; and (iv) applying graph database algorithms to ascertain relationships represented by the edges between entries in the medical data; and
create a visual output comprising (i) a health snapshot containing automatically generated prose describing the patient's health based on the knowledge graph and ranked using a page rank algorithm, (ii) a body map identifying areas of the patient's body having health issues based on the knowledge graph, wherein the body map includes interactive indicators that, upon user selection, reveal detailed health information extracted from the knowledge graph, and (iii) a health history displayed in an enhanced timeline format with interactive elements allowing drill-down into specific health events.

2. The system of claim 1, wherein the visual output further comprises information relating to a prediction generated by the artificial intelligence model based on analysis of the knowledge graph.

3. The system of claim 1, wherein the platform is further configured to calculate and display a health score using the knowledge graph and the artificial intelligence model.

4. The system of claim 1, wherein the knowledge graph includes nodes representing prescribed medications and negative drug outcomes, and wherein the artificial intelligence model analyzes relationships between these nodes to identify drug-related risk factors.

5. The system of claim 1, wherein the medical data further comprises a medical image, and wherein the knowledge graph includes nodes representing image features and diagnostic correlations identified by the artificial intelligence model.

6. The system of claim 1, wherein generating the knowledge graph further comprises identifying the data elements as nodes by extracting patient medical information relating to organizations, payors, and practitioners associated with the patient.

7. The system of claim 1, wherein the page rank algorithm ranks health information based on how frequently it is referenced by other health records in the medical data.

8. The system of claim 1, wherein the interactive indicators on the body map are displayed as dots on specific body parts, and wherein selecting an indicator reveals detailed health information for the corresponding body part.

9. The system of claim 1, wherein the artificial intelligence model is configured to analyze medical images to identify features correlated with specific diagnoses based on historical patient data.

10. The system of claim 1, wherein the platform is further configured to:
generate a confidence score for predicted diagnoses based on similarity determinations between features identified in current medical data and correlated features from historical patient data.

11. A computer-implemented method comprising:
receiving medical data relating to a patient in a Fast Healthcare Interoperability Resources (FHIR) or Longitudinal Patient Records (LPR) format, wherein the medical data comprises a patient record and at least one vital statistic;
performing temporal analysis on the medical data to produce temporal analysis data;
extract features of the medical data to produce features data;
using an artificial intelligence model trained on historical patient data, analyzing the medical data, the temporal analysis data and the features data to generate a knowledge graph by: (i) identifying data elements as nodes in the knowledge graph, (ii) determining relationships between the data elements using semantic analysis and natural language processing of the medical data, (iii) generating edges between the nodes based on the determined relationships; and (iv) applying graph database algorithms to ascertain relationships represented by the edges between entries in the medical data; and
creating a visual output comprising (i) a health snapshot containing automatically generated prose describing the patient's health based on the knowledge graph and ranked using a page rank algorithm, (ii) a body map identifying areas of the patient's body having health issues based on the knowledge graph, wherein the body map includes interactive indicators that, upon user selection, reveal detailed health information extracted from the knowledge graph, and (iii) a health history displayed in an enhanced timeline format with interactive elements allowing drill-down into specific health events.

12. The method of claim 11, wherein the visual output further comprises information relating to a prediction generated by the artificial intelligence model based on analysis of the knowledge graph.

13. The method of claim 11, further comprising:
calculating and displaying a health score using the knowledge graph and the artificial intelligence model.

14. The method of claim 11, wherein the knowledge graph includes nodes representing prescribed medications and negative drug outcomes, and wherein the artificial intelligence model analyzes relationships between these nodes to identify drug-related risk factors.

15. The method of claim 11, wherein the medical data further comprises a medical image, and wherein the knowledge graph includes nodes representing image features and diagnostic correlations identified by the artificial intelligence model.

16. The method of claim 11, wherein generating the knowledge graph further comprises identifying the data elements as nodes by extracting patient medical information relating to organizations, payors, and practitioners associated with the patient.

17. The method of claim 11, wherein the page rank algorithm ranks health information based on how frequently it is referenced by other health records in the medical data.

18. The method of claim 11, wherein the interactive indicators on the body map are displayed as dots on specific body parts, and wherein selecting an indicator reveals detailed health information for the corresponding body part.

19. The method of claim 11, wherein the artificial intelligence model analyzes medical images to identify features correlated with specific diagnoses based on historical patient data.

20. The method of claim 11, further comprising:
generating a confidence score for predicted diagnoses based on similarity determinations between features identified in current medical data and correlated features from historical patient data.
